(19) United States
(12) Patent Application Publication
(10) Pub. No.: US 2011/0212944 A1
(43) Pub. Date: Sep. 1, 2011

(54) 2-OXO-1-PYRROLIDINE DERIVATIVES

(76) Inventors: Julie Liu, Lexington, MA (US); Rose Persichetti, Stow, MA (US)

(21) Appl. No.: 13/002,267

(22) PCT Filed: Jun. 30, 2009
(86) PCT No.: PCT/US09/49224

§ 371 Date: May 4, 2011

Related U.S. Application Data
(60) Provisional application No. 61/077,349, filed on Jul. 1, 2008.

(51) Int. Cl. A61K 31/55; C07D 207/26; A61K 31/4015; A61K 31/53; A61K 31/4166; A61P 25/00; A61P 11/00; A61P 13/00; A61P 25/04

(52) U.S. Cl. 514/217; 548/550; 514/424; 514/242; 514/391

(57) ABSTRACT

This invention relates to novel 2-oxo-1-pyrrolidines, their derivatives, and pharmaceutically acceptable salts thereof. This invention also provides compositions comprising a compound of this invention and the use of such compositions in methods of treating diseases and conditions that are beneficially treated by administering a compound with the ability to act as a synaptic vesicle protein 2A (SV2A) ligand and/or a sodium channel blocker.

### CROSS REFERENCE TO RELATED APPLICATIONS

This application claims priority to U.S. Application No. 61/077,349, filed Jul. 1, 2008, which is incorporated herein by reference in its entirety.

### FIELD OF THE INVENTION

This invention relates to deuterated derivatives of 2-oxo-1-pyrrolidines, and pharmaceutically acceptable salts thereof. This invention also provides compositions comprising a compound of this invention and the use of such compositions in methods of treating diseases and conditions that are beneficially treated by administering a compound with the ability to act as a synaptic vesicle protein 2A (SV2A) ligand and/or a sodium channel blocker.

### BACKGROUND OF THE INVENTION

Brivaracetam, also known as 2(S)-[2-oxo-4(R)-propylpyrrolidin-1-yl]butyramide, binds to synaptic vesicle protein 2A (SV2A) and inhibits activity at neuronal voltage-dependent sodium channels in correlation with suppression of seizures due to acquired or genetic epilepsy (von Rosenstiel, P., Neurotherapeutics, 2007, Jan., 4(1): 84-7).

Brivaracetam is currently undergoing clinical trials for the treatment of epilepsy in refractory patients with partial onset seizures, for Unverricht-Lundborg Disease (ULD) also known as progressive myoclonic epilepsy Type 1 (EPM1) and for tremor. Brivaracetam has received orphan drug status in the US and EU for the treatment of symptomatic myoclonic epilepsies.

Treatment emergent adverse events related to brivaracetam were mild to moderate in severity and primarily CNS-related. These events include, but are not limited to, dizziness, somnolence, headache, euphoric mood, throat irritation, fatigue/nausea and blurred vision/feeling drunk/agitation/hypotension, all of which subsided within 24 hours of drug administration. (Rola, P. et al., Epilepsia, 2004, 45(suppl 7): abstract 2.365).

Despite the beneficial activities of brivaracetam, there is a continuing need for new compounds to treat the aforementioned diseases and conditions.

### SUMMARY OF THE INVENTION

The invention provides a compound of Formula I:

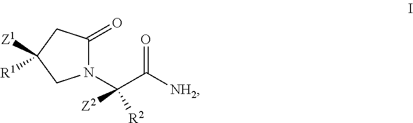

or a pharmaceutically acceptable salt thereof, wherein each Z is independently selected from hydrogen and deuterium, R¹ is an n-propyl group having zero to seven deuterium atoms, R² is an ethyl group having zero to five deuterium atoms; and when each R has zero deuterium atoms, at least one Z is deuterium.

In an embodiment of the invention, R¹ is selected from CD₃CH₂CH₂—, CD₃CD₂CH₂—, CD₃CH₂CD₂-, CH₃CH₂CD₂-, CH₃CD₂CD₂-, CD₃CD₂CD₂- and CH₃CH₂CH₂—.

In another embodiment of the invention, R² is selected from CH₃CH₂—, CD₃CH₂—, CH₃CD₂-, and CD₃CD₂-.

In another embodiment of the invention, R¹ is CD₃CH₂CH₂—, or CD₃CD₂CH₂—, and R² is CH₃CH₂—, CD₃CH₂—, CH₃CD₂-, or CD₃CD₂-.

In particular embodiments of the invention, the compound is

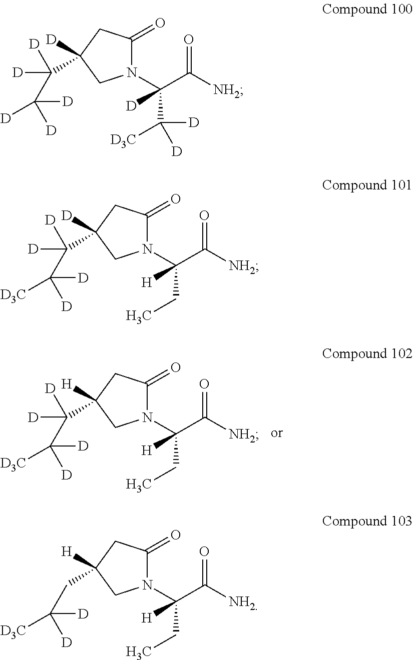

According to the invention, in compounds of Formula I, any atom not designated as deuterium is present at its natural isotopic abundance.

The invention further provides a composition comprising a compound of Formula I and an acceptable carrier. In an embodiment of the invention, the composition is pyrogen free. When the compound is formulated for pharmaceutical administration, the carrier is a pharmaceutically acceptable carrier.

The composition comprising Formula I can further include a second therapeutic agent useful in the treatment of a patient suffering from or susceptible to a disease or condition selected from epilepsy, epileptogenesis, seizure disorders, convulsions, bipolar disorders, mania, depression, anxiety, migraine, trigeminal and other neuralgia, chronic pain, neuropathic pain, cerebral ischemia, cardiac arrhythmia, myotonia, cocaine abuse, stroke, myoclonus, essential tremor and other movement disorders, neonatal cerebral hemorrhage, amyotrophic lateral sclerosis, spasticity, Parkinson's disease and other degenerative diseases, bronchial asthma, asthmatic status and allergic bronchitis, asthmatic syndrome, bronchial hyperreactivity, bronchospastic syndromes, allergic and vasomotor rhinitis, rhinoconjunctivitis, lower urinary tract dysfunction including urinary incontinence and overactive bladder, fecal incontinence, conditions of lower urinary tract dysfunction related to benign prostatic hyperplasia and fecal incontinence, and progressive myoclonic epilepsies (PME) including Unverricht-Lundborg disease, Lafora disease, myoclonic epilepsy with ragged red fibres, neuronal ceroid lipofuscinosis, sialidoses, and dentatorubral-pallidoluysian atrophy. Accordingly, in an embodiment of the invention, the second therapeutic agent is carbamazepine, lamotrigine, levetiracetam, oxcarbazepine, phenyloin, topiramate, or valproate.

The invention provides a method of modulating the activity of synaptic vesicle protein 2A (SV2A) and/or inhibiting activity at neuronal voltage-dependent sodium channels in a cell of the central nervous system, comprising contacting the cell with a compound of formula I.

The invention also provides a method for treating or ameliorating a disease in a subject comprising administering an effective amount of a compound of formula I and a pharmaceutically acceptable carrier. Subjects to be treated include patients suffering from, or susceptible to, a disease or condition selected from epilepsy, epileptogenesis, seizure disorders, convulsions, bipolar disorders, mania, depression, anxiety, migraine, trigeminal and other neuralgia, chronic pain, neuropathic pain, cerebral ischemia, cardiac arrhythmia, myotonia, cocaine abuse, stroke, myoclonus, essential tremor and other movement disorders, neonatal cerebral hemorrhage, amyotrophic lateral sclerosis, spasticity, Parkinson's disease and other degenerative diseases, bronchial asthma, asthmatic status and allergic bronchitis, asthmatic syndrome, bronchial hyperreactivity, bronchospastic syndromes, allergic and vasomotor rhinitis, rhinoconjunctivitis, lower urinary tract dysfunction including urinary incontinence and overactive bladder, fecal incontinence, conditions of lower urinary tract dysfunction related to benign prostatic hyperplasia and fecal incontinence, and progressive myoclonic epilepsies (PME) including Unverricht-Lundborg disease, Lafora disease, myoclonic epilepsy with ragged red fibres, neuronal ceroid lipofuscinosis, sialidoses, and dentatorubral-pallidoluysian atrophy.

In a particular embodiment, the patient is suffering from or susceptible to a disease or condition selected from epilepsy, Unverricht-Lundborg Disease (ULD; also known as progressive myoclonic epilepsy Type 1 (EPM1)), tremor and symptomatic myoclonic epilepsies.

According to the invention, a compound of formula I may be coadministered to a patient in need thereof with a second therapeutic agent useful in treating or preventing a disease or condition selected from epilepsy, epileptogenesis, seizure disorders, convulsions, bipolar disorders, mania, depression, anxiety, migraine, trigeminal and other neuralgia, chronic pain, neuropathic pain, cerebral ischemia, cardiac arrhythmia, myotonia, cocaine abuse, stroke, myoclonus, essential tremor and other movement disorders, neonatal cerebral hemorrhage, amyotrophic lateral sclerosis, spasticity, Parkinson's disease and other degenerative diseases, bronchial asthma, asthmatic status and allergic bronchitis, asthmatic syndrome, bronchial hyperreactivity, bronchospastic syndromes, allergic and vasomotor rhinitis, rhinoconjunctivitis, lower urinary tract dysfunction including urinary incontinence and overactive bladder, fecal incontinence, conditions of lower urinary tract dysfunction related to benign prostatic hyperplasia and fecal incontinence, and progressive myoclonic epilepsies (PME) including Unverricht-Lundborg disease, Lafora disease, myoclonic epilepsy with ragged red fibres, neuronal ceroid lipofuscinosis, sialidoses, and dentatorubral-pallidoluysian atrophy.

In an embodiment of the invention, wherein the patient is suffering from or susceptible to epilepsy, the second therapeutic agent is selected from carbamazepine, lamotrigine, levetiracetam, oxcarbazepine, phenyloin, topiramate, and valproate.

### DETAILED DESCRIPTION OF THE INVENTION

The term “treat” means decrease, suppress, attenuate, diminish, arrest, or stabilize the development or progression of a disease (e.g., a disease or disorder delineated herein). They should not be taken to imply that a subject is treated to total recovery.

“Disease” means any condition or disorder that damages or interferes with the normal function of a cell, tissue, or organ.

It will be recognized that some variation of natural isotopic abundance occurs in a synthesized compound depending upon the origin of chemical materials used in the synthesis. Thus, a preparation of brivaracetam will inherently contain small amounts of deuterated isotopologues. The concentration of naturally abundant stable hydrogen and carbon isotopes, notwithstanding this variation, is small and immaterial as compared to the degree of stable isotopic substitution of compounds of this invention. See, e.g., Wada, E. et al., Seikagaku 1994, 66:15; Gannes, L. Z. et al., Comp. Biochem. Physiol. Mol. Integr. Physiol. 1998, 119:725.

In the compounds of this invention any atom not specifically designated as a particular isotope is meant to represent any stable isotope of that atom. In a compound of this invention, when a particular position is designated as having deuterium, it is understood that the abundance of deuterium at that position is substantially greater than the natural abundance of deuterium, which is 0.015%. A position designated as having deuterium has a minimum isotopic enrichment factor of at least about 3000 (45% deuterium incorporation) at each atom designated as deuterium in said compound.

The term “isotopic enrichment factor” as used herein means the ratio between the isotopic abundance and the natural abundance of a specified isotope.

In other embodiments, a compound of this invention has an isotopic enrichment factor for each designated deuterium atom of at least about 3500 (52.5% deuterium incorporation at each designated deuterium atom), at least about 4000 (60% deuterium incorporation), at least about 4500 (67.5% deuterium incorporation), at least about 5000 (75% deuterium incorporation), at least about 5500 (82.5% deuterium incorporation), at least about 6000 (90% deuterium incorporation), at least about 6333.3 (95% deuterium incorporation), at least about 6466.7 (97% deuterium incorporation), at least about 6600 (99% deuterium incorporation), or at least about 6633.3 (99.5% deuterium incorporation). It is understood that the isotopic enrichment factor of each deuterium present at a site designated as a site of deuteration is independent of other deuterated sites. For example, if there are two sites of deuteration on a compound one site could be deuterated at 52.5% while the other could be deuterated at 75%. The resulting compound would be considered to be a compound wherein the isotopic enrichment factor is at least 3500 (52.5%).

The term “isotopologue” refers to a species that differs from a specific compound of this invention only in the isotopic composition thereof. The term “compound,” as used herein, refers to a collection of molecules having an identical chemical structure, except that there may be isotopic variation among the constituent atoms of the molecules. Thus, it will be clear to those of skill in the art that a compound represented by a particular chemical structure containing indicated deuterium atoms, will also contain lesser amounts of isotopologues having hydrogen atoms at one or more of the designated deuterium positions in that structure. The relative amount of such isotopologues in a compound of this invention will depend upon a number of factors including the isotopic purity of deuterated reagents used to make the compound and the efficiency of incorporation of deuterium in the various synthesis steps used to prepare the compound. However, as set forth above the relative amount of such isotopologues will be less than 49.9% of the compound. In other embodiments, the relative amount of such isotopologues in toto will be less than 47.5%, less than 40%, less than 32.5%, less than 25%, less than 17.5%, less than 10%, less than 5%, less than 3%, less than 1%, or less than 0.5% of the compound.

The invention also provides salts of the compounds of the invention.

A salt of a compound of this invention is formed between an acid and a basic group of the compound, such as an amino functional group, or a base and an acidic group of the compound, such as a carboxyl functional group. According to another embodiment, the compound is a pharmaceutically acceptable acid addition salt.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this invention. A “pharmaceutically acceptable counterion” is an ionic portion of a salt that is not toxic when released from the salt upon administration to a recipient.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

The compounds of the present invention (e.g., compounds of Formula I), may contain an asymmetric carbon atom, for example, as the result of deuterium substitution or otherwise. As such, compounds of this invention can exist as either individual enantiomers, or mixtures of the two enantiomers. Accordingly, a compound of the present invention will include both racemic mixtures, and also individual respective stereoisomers that are substantially free from another possible stereoisomer. The term “substantially free of other stereoisomers” as used herein means less than 25% of other stereoisomers, preferably less than 10% of other stereoisomers, more preferably less than 5% of other stereoisomers and most preferably less than 2% of other stereoisomers, or less than “X”% of other stereoisomers (wherein X is a number between 0 and 100, inclusive) are present. Methods of obtaining or synthesizing an individual enantiomer for a given compound are well known in the art and may be applied as practicable to final compounds or to starting material or intermediates.

The term “stable compounds,” as used herein, refers to compounds which possess stability sufficient to allow for their manufacture and which maintain the integrity of the compound for a sufficient period of time to be useful for the purposes detailed herein (e.g., formulation into therapeutic products, intermediates for use in production of therapeutic compounds, isolatable or storable intermediate compounds, treating a disease or condition responsive to therapeutic agents).

“D” refers to deuterium. “Stereoisomer” refers to both enantiomers and diastereomers. “Tert”, “^{t}”, and “t-” each refer to tertiary. “US” refers to the United States of America.

Throughout this specification, a variable may be referred to generally (e.g., “each R”) or may be referred to specifically (e.g., R¹, R², R³, etc.). Unless otherwise indicated, when a variable is referred to generally, it is meant to include all specific embodiments of that particular variable.

### Therapeutic Compounds

The present invention provides a compound of Formula I:

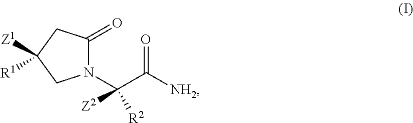

or a pharmaceutically acceptable salt thereof, wherein:

each Z is independently selected from hydrogen and deuterium;

R¹ is an n-propyl group having zero to seven deuterium atoms;

R² is an ethyl group having zero to five deuterium atoms, and

when each R has zero deuterium atoms, at least one Z is deuterium.

One embodiment of this invention provides compounds of Formula I wherein R¹ is selected from CD₃CH₂CH₂—, CD₃CD₂CH₂—, CD₃CH₂CD₂-, CH₃CH₂CD₂-, CH₃CD₂CD₂-, CD₃CD₂CD₂- or CH₃CH₂CH₂—. In a more specific embodiment, R¹ is CD₃CD₂CD₂- or CD₃CD₂CH₂—. In one aspect of these embodiments, Z¹ and Z² are both hydrogen. In another aspect of these embodiments, Z¹ and Z² are both deuterium.

In another embodiment, R² is selected from CH₃CH₂—, CD₃CH₂—, CH₃CD₂-, or CD₃CD₂-. In a more specific embodiment, R² is selected from CH₃CH₂— or CD₃CD₂-. In one aspect of these embodiments, Z¹ and Z² are both hydrogen. In another aspect of these embodiments, Z¹ and Z² are both deuterium.

The R and Z variables as described above may be selected and taken together to provide more specific embodiments of this invention. For example, in one embodiment, R¹ is CD₃CH₂CH₂—, CD₃CD₂CH₂—, CD₃CH₂CD₂-, CH₃CH₂CD₂-, CH₃CD₂CD₂-, CD₃CD₂CD₂- or CH₃CH₂CH₂—; and R² is selected from CH₃CH₂—, CD₃CH₂—, CH₃CD₂-, or CD₃CD₂-. In one aspect of this embodiment, R² is CH₃CH₂— or CD₃CD₂-.

In another embodiment, R¹ is CD₃CD₂CD₂- or CD₃CD₂CH₂—; and R² is selected from CH₃CH₂—, CD₃CH₂—, CH₃CD₂-, or CD₃CD₂-. In one aspect of this embodiment, R² is CH₃CH₂— or CD₃CD₂-.

Examples of specific compounds of this invention include the following:

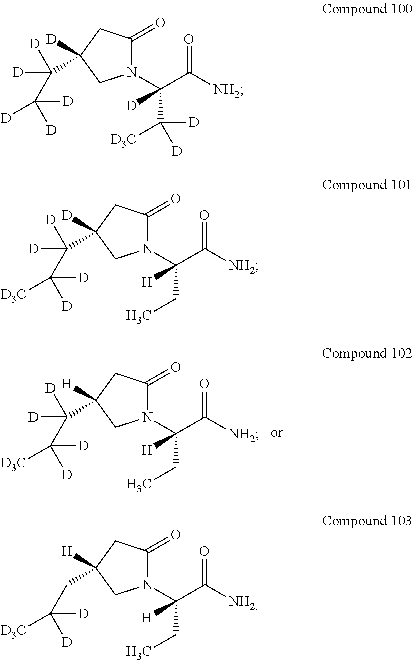

In another set of embodiments, any atom not designated as deuterium in any of the embodiments set forth above is present at its natural isotopic abundance.

The synthesis of compounds of Formula I may be achieved by synthetic chemists of ordinary skill. Relevant procedures and intermediates are disclosed, for instance in PCT patent publications WO2007065634, WO2005028435, and WO 2001062726; and in Kenda, B. M. et al., J. Med. Chem. 2004, 47(3): 530-549.

Such methods can be carried out utilizing corresponding deuterated and optionally, other isotope-containing reagents and/or intermediates to synthesize the compounds delineated herein, or invoking standard synthetic protocols known in the art for introducing isotopic atoms to a chemical structure.

### Exemplary Synthesis

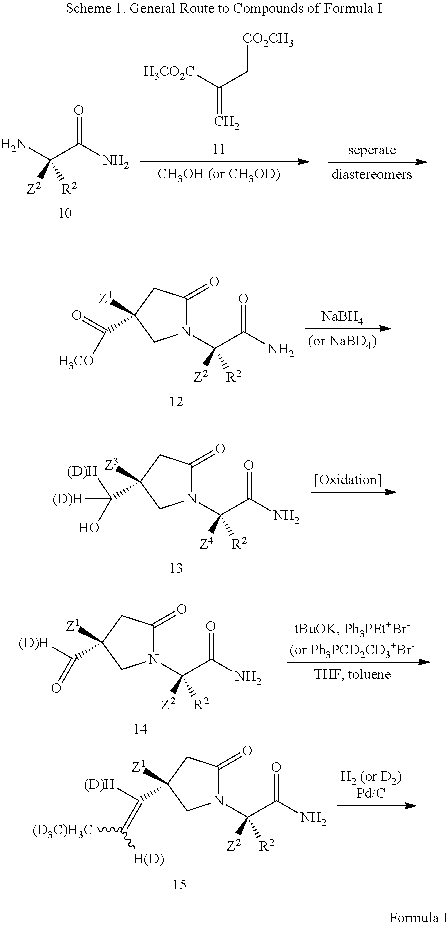

Scheme I above shows a general route for preparing compounds of Formula I. According to the general methods of Kenda, B. M. et al, J. Med. Chem. 2004, 47(3): 530-549, an appropriately-deuterated amide 10 is cyclized with ester 11 in either CH₃OH or CH₃OD to afford a mixture of separable diastereomeric esters. Ester 12 is isolated and reduced with either NaBH₄ or NaBD₄ to provide alcohol 13. Oxidation to aldehyde 14 is achieved through one of many known general methods of oxidation, such as Swern oxidation or Dess-Martin reagent or sulfur trioxide-pyridine. Wittig reaction of aldehyde 14 with either commercially-available ethyltriphenylphosphonium bromide or known (ethyl-d5)triphenylphosphonium bromide (Schultz, M J et al, J Am Chem Soc 2006, 1460-1461) in the presence of tBuOK affords olefin 15, according to the general method of Maring, C J et al, J Med Chem 2005, 48(12):3980-3990. Alternatively, the Wittig reaction may be conducted using nBuLi instead of tBuOK, according to the general method of Gil, A M et al, Tet Asym 2003, 14(11):1479-1488. Reduction of the olefin 15 using either H₂ or D₂ in the presence of palladium on carbon provides a compound of Formula I.

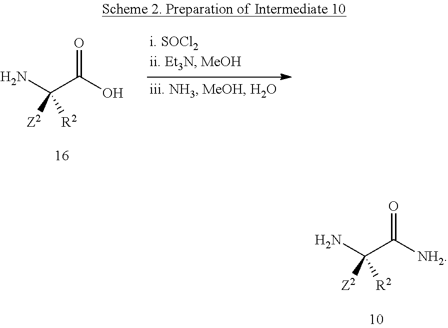

Scheme 2 depicts the preparation of deuterated amide 10. An appropriately-deuterated carboxylic acid 16 is converted to amide 10 by treatment with thionyl chloride, followed by triethylamine and methanol, then followed by ammonia, according to the methods of Zhou, X et al, Jingxi Huagong Zhongjianti 2005, 35(2):27-28. Appropriately-deuterated carboxylic acids 16 are known, some of which are commercially available. These include commercially available (2S)-2-aminobutanoic acid-d3,

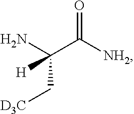

and L-2-aminobutyric-3,3-d2 acid,

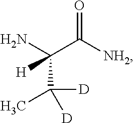

as well as known (S)-2-aminobutanoic-2-dl acid,

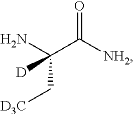

(Milne, J. J. et al., Biochem. Soc. Trans. 1996, 24(1):133S], and (S)-2-aminobutanoic-2,3,3-d3 acid,

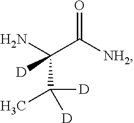

(Esaki, N. et al., Anal. Biochem. 1982, 119(2):281-5).

The specific approaches and compounds shown above are not intended to be limiting. The chemical structures in the schemes herein depict variables that are hereby defined commensurately with chemical group definitions (moieties, atoms, etc.) of the corresponding position in the compound formulae herein, whether identified by the same variable name (i.e., R¹, R², Z¹, etc.) or not. The suitability of a chemical group in a compound structure for use in the synthesis of another compound is within the knowledge of one of ordinary skill in the art.

Additional methods of synthesizing compounds of Formula I and their synthetic precursors, including those within routes not explicitly shown in schemes herein, are within the means of chemists of ordinary skill in the art. Synthetic chemistry transformations and protecting group methodologies (protection and deprotection) useful in synthesizing the applicable compounds are known in the art and include, for example, those described in Larock R, Comprehensive Organic Transformations, VCH Publishers (1989); Greene T W et al., Protective Groups in Organic Synthesis, 3^{rd} Ed., John Wiley and Sons (1999); Fieser L et al., Fieser and Fieser's Reagents for Organic Synthesis, John Wiley and Sons (1994); and Paquette L, ed., Encyclopedia of Reagents for Organic Synthesis, John Wiley and Sons (1995) and subsequent editions thereof.

Combinations of substituents and variables envisioned by this invention are only those that result in the formation of stable compounds.

### Compositions

The invention also provides pyrogen-free compositions comprising an effective amount of a compound of Formula I (e.g., including any of the formulae herein), or a pharmaceutically acceptable salt of said compound; and an acceptable carrier. Preferably, a composition of this invention is formulated for pharmaceutical use (“a pharmaceutical composition”), wherein the carrier is a pharmaceutically acceptable carrier. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

If required, the solubility and bioavailability of the compounds of the present invention in pharmaceutical compositions may be enhanced by methods well-known in the art. One method includes the use of lipid excipients in the formulation. See “Oral Lipid-Based Formulations Enhancing the Bioavailability of Poorly Water-Soluble Drugs (Drugs and the Pharmaceutical Sciences),” David J. Hauss, ed. Informa Healthcare, 2007; and “Role of Lipid Excipients in Modifying Oral and Parenteral Drug Delivery: Basic Principles and Biological Examples,” Kishor M. Wasan, ed. Wiley-Interscience, 2006.

Another known method of enhancing bioavailability is the use of an amorphous form of a compound of this invention optionally formulated with a poloxamer, such as LUTROL™ and PLURONIC™ (BASF Corporation), or block copolymers of ethylene oxide and propylene oxide. See U.S. Pat. No. 7,014,866; and United States patent publications 2006/0094744 and 2006/0079502.

The pharmaceutical compositions of the invention include those suitable for oral, rectal, nasal, topical (including buccal and sublingual), vaginal or parenteral (including subcutaneous, intramuscular, intravenous and intradermal) administration. In certain embodiments, the compound of the formulae herein is administered transdermally (e.g., using a transdermal patch or iontophoretic techniques). Other formulations may conveniently be presented in unit dosage form, e.g., tablets, sustained release capsules, and in liposomes, and may be prepared by any methods well known in the art of pharmacy. See, for example, Remington's Pharmaceutical Sciences, Mack Publishing Company, Philadelphia, Pa. (17th ed. 1985).

Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In certain embodiments, the compound is administered orally. Compositions of the present invention suitable for oral administration may be presented as discrete units such as capsules, sachets, or tablets each containing a predetermined amount of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption.

In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added.

Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Such injection solutions may be in the form, for example, of a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of this invention may be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, e.g.: Rabinowitz J D and Zaffaroni A C, U.S. Pat. No. 6,803,031, assigned to Alexza Molecular Delivery Corporation.

Topical administration of the pharmaceutical compositions of this invention is especially useful when the desired treatment involves areas or organs readily accessible by topical application. For topical application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax, and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol, and water. The pharmaceutical compositions of this invention may also be topically applied to the lower intestinal tract by rectal suppository formulation or in a suitable enema formulation. Topically-transdermal patches and iontophoretic administration are also included in this invention.

Application of the subject therapeutics may be local, so as to be administered at the site of interest. Various techniques can be used for providing the subject compositions at the site of interest, such as injection, use of catheters, trocars, projectiles, pluronic gel, stents, sustained drug release polymers or other device which provides for internal access.

Thus, according to yet another embodiment, the compounds of this invention may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the invention provides a method of coating an implantable medical device comprising the step of contacting said device with the coating composition described above. It will be obvious to those skilled in the art that the coating of the device will occur prior to implantation into a mammal.

According to another embodiment, the invention provides a method of impregnating an implantable drug release device comprising the step of contacting said drug release device with a compound or composition of this invention. Implantable drug release devices include, but are not limited to, biodegradable polymer capsules or bullets, non-degradable, diffusible polymer capsules and biodegradable polymer wafers.

According to another embodiment, the invention provides an implantable medical device coated with a compound or a composition comprising a compound of this invention, such that said compound is therapeutically active.

According to another embodiment, the invention provides an implantable drug release device impregnated with or containing a compound or a composition comprising a compound of this invention, such that said compound is released from said device and is therapeutically active.

Where an organ or tissue is accessible because of removal from the patient, such organ or tissue may be bathed in a medium containing a composition of this invention, a composition of this invention may be painted onto the organ, or a composition of this invention may be applied in any other convenient way.

In another embodiment, a composition of this invention further comprises a second therapeutic agent. The second therapeutic agent may be selected from any compound or therapeutic agent known to have or that demonstrates advantageous properties when administered with a compound having the same mechanism of action as brivaracetam. Such agents include those indicated as being useful in combination with brivaracetam.

Preferably, the second therapeutic agent is an agent useful in the treatment or prevention of a disease or condition selected from epilepsy, epileptogenesis, seizure disorders, convulsions, bipolar disorders, mania, depression, anxiety, migraine, trigeminal and other neuralgia, chronic pain, neuropathic pain, cerebral ischemia, cardiac arrhythmia, myotonia, cocaine abuse, stroke, myoclonus, essential tremor and other movement disorders, neonatal cerebral hemorrhage, amyotrophic lateral sclerosis, spasticity, Parkinson's disease and other degenerative diseases, bronchial asthma, asthmatic status and allergic bronchitis, asthmatic syndrome, bronchial hyperreactivity, bronchospastic syndromes, allergic and vasomotor rhinitis, rhinoconjunctivitis, lower urinary tract dysfunction including urinary incontinence and overactive bladder, fecal incontinence, conditions of lower urinary tract dysfunction related to benign prostatic hyperplasia and fecal incontinence, and progressive myoclonic epilepsies (PME) including Unverricht-Lundborg disease, Lafora disease, myoclonic epilepsy with ragged red fibres, neuronal ceroid lipofuscinosis, sialidoses, and dentatorubral-pallidoluysian atrophy.

In one embodiment, the second therapeutic agent is selected from carbamazepine, lamotrigine, levetiracetam, oxcarbazepine, phenyloin, topiramate, and valproate.

In another embodiment, the invention provides separate dosage forms of a compound of this invention and one or more of any of the above-described second therapeutic agents, wherein the compound and second therapeutic agent are associated with one another. The term “associated with one another” as used herein means that the separate dosage forms are packaged together or otherwise attached to one another such that it is readily apparent that the separate dosage forms are intended to be sold and administered together (within less than 24 hours of one another, consecutively or simultaneously).

In the pharmaceutical compositions of the invention, the compound of the present invention is present in an effective amount. As used herein, the term “effective amount” refers to an amount which, when administered in a proper dosing regimen, is sufficient to reduce or ameliorate the severity, duration or progression of the disorder being treated, prevent the advancement of the disorder being treated, cause the regression of the disorder being treated, or enhance or improve the prophylactic or therapeutic effect(s) of another therapy.

The interrelationship of dosages for animals and humans (based on milligrams per meter squared of body surface) is described in Freireich et al., (1966) Cancer Chemother. Rep 50: 219. Body surface area may be approximately determined from height and weight of the patient. See, e.g., Scientific Tables, Geigy Pharmaceuticals, Ardsley, N.Y., 1970, 537.

In one embodiment, an effective amount of a compound of this invention can range from about 0.025 mg to 10,000 mg per treatment. In a more specific embodiment the range is from about 0.25 mg to 5000 mg, or from about 0.5 mg to 2000 mg, or most specifically from about 2.5 mg to about 1000 mg per treatment. Treatment typically is administered one to two times daily.

Effective doses will also vary, as recognized by those skilled in the art, depending on the diseases treated, the severity of the disease, the route of administration, the sex, age and general health condition of the patient, excipient usage, the possibility of co-usage with other therapeutic treatments such as use of other agents and the judgment of the treating physician. For example, guidance for selecting an effective dose can be determined by reference to the prescribing information for brivaracetam.

For pharmaceutical compositions that comprise a second therapeutic agent, an effective amount of the second therapeutic agent is between about 20% and 100% of the dosage normally utilized in a monotherapy regime using just that agent. Preferably, an effective amount is between about 70% and 100% of the normal monotherapeutic dose. The normal monotherapeutic dosages of these second therapeutic agents are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are incorporated herein by reference in their entirety.

It is expected that combinations with second therapeutic agents referenced above will provide improved therapeutic responses. When this occurs, it will allow the effective dosage of the second therapeutic agent and/or the compound of this invention to be reduced from that required in a monotherapy. This has the advantage of minimizing toxic side effects of either the second therapeutic agent of a compound of this invention, additive or synergistic improvements in efficacy, improved ease of administration or use and/or reduced overall expense of compound preparation or formulation.

### Methods of Treatment

In another embodiment, the invention provides a method of modulating the activity of synaptic vesicle protein 2A (SV2A) and/or inhibiting activity at neuronal voltage-dependent sodium channels in a cell of the central nervous system, comprising contacting such a cell with one or more compounds of Formula I herein or a pharmaceutically acceptable salt thereof.

According to another embodiment, the invention provides a method of treating a patient suffering from, or susceptible to, a disease that is beneficially treated by brivaracetam comprising the step of administering to said patient an effective amount of a compound of Formula I herein or a pharmaceutically acceptable salt thereof, or a composition of this invention. Such diseases are well known in the art and are disclosed in, but not limited to the following patents and published applications: WO 2001062726, WO 2006090275, and WO 2006131322.

Such diseases include, but are not limited to, epilepsy, epileptogenesis, seizure disorders, convulsions, bipolar disorders, mania, depression, anxiety, migraine, trigeminal and other neuralgia, chronic pain, neuropathic pain, cerebral ischemia, cardiac arrhythmia, myotonia, cocaine abuse, stroke, myoclonus, essential tremor and other movement disorders, neonatal cerebral hemorrhage, amyotrophic lateral sclerosis, spasticity, Parkinson's disease and other degenerative diseases, bronchial asthma, asthmatic status and allergic bronchitis, asthmatic syndrome, bronchial hyperreactivity, bronchospastic syndromes, allergic and vasomotor rhinitis, rhinoconjunctivitis, lower urinary tract dysfunction including urinary incontinence and overactive bladder, fecal incontinence, conditions of lower urinary tract dysfunction related to benign prostatic hyperplasia and fecal incontinence, and progressive myoclonic epilepsies (PME) including Unverricht-Lundborg disease, Lafora disease, myoclonic epilepsy with ragged red fibres, neuronal ceroid lipofuscinosis, sialidoses, and dentatorubral-pallidoluysian atrophy.

In one particular embodiment, the method of this invention is used to treat a patient suffering from or susceptible to a disease or condition selected from epilepsy, Unverricht-Lundborg Disease (ULD) also known as progressive myoclonic epilepsy Type 1 (EPM1), tremor and symptomatic myoclonic epilepsies.

Methods delineated herein also include those wherein the patient is identified as in need of a particular stated treatment. Identifying a patient in need of such treatment can be in the judgment of a patient or a health care professional and can be subjective (e.g. opinion) or objective (e.g. measurable by a test or diagnostic method).

In another embodiment, any of the above methods of treatment comprises the further step of co-administering to the patient one or more second therapeutic agents. The choice of second therapeutic agent may be made from any second therapeutic agent known to be useful for co-administration with brivaracetam. The choice of second therapeutic agent is also dependent upon the particular disease or condition to be treated. Examples of second therapeutic agents that may be employed in the methods of this invention are those set forth above for use in combination compositions comprising a compound of this invention and a second therapeutic agent.

In particular, the combination therapies of this invention include co-administering a compound of Formula I or a pharmaceutically acceptable salt thereof and a second therapeutic agent for treatment of the following conditions (with the particular second therapeutic agent indicated in parentheses following the indication: epilepsy (carbamazepine, lamotrigine, levetiracetam, oxcarbazepine, phenyloin, topiramate, and valproate).

The term “co-administered” as used herein means that the second therapeutic agent may be administered together with a compound of this invention as part of a single dosage form (such as a composition of this invention comprising a compound of the invention and an second therapeutic agent as described above) or as separate, multiple dosage forms. Alternatively, the additional agent may be administered prior to, consecutively with, or following the administration of a compound of this invention. In such combination therapy treatment, both the compounds of this invention and the second therapeutic agent(s) are administered by conventional methods. The administration of a composition of this invention, comprising both a compound of the invention and a second therapeutic agent, to a patient does not preclude the separate administration of that same therapeutic agent, any other second therapeutic agent or any compound of this invention to said patient at another time during a course of treatment.

Effective amounts of these second therapeutic agents are well known to those skilled in the art and guidance for dosing may be found in patents and published patent applications referenced herein, as well as in Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), and other medical texts. However, it is well within the skilled artisan's purview to determine the second therapeutic agent's optimal effective-amount range.

In one embodiment of the invention, where a second therapeutic agent is administered to a subject, the effective amount of the compound of this invention is less than its effective amount would be where the second therapeutic agent is not administered. In another embodiment, the effective amount of the second therapeutic agent is less than its effective amount would be where the compound of this invention is not administered. In this way, undesired side effects associated with high doses of either agent may be minimized. Other potential advantages (including without limitation improved dosing regimens and/or reduced drug cost) will be apparent to those of skill in the art.

In yet another aspect, the invention provides the use of a compound of Formula I alone or together with one or more of the above-described second therapeutic agents in the manufacture of a medicament, either as a single composition or as separate dosage forms, for treatment or prevention in a patient of a disease, disorder or symptom set forth above. Another aspect of the invention is a compound of Formula I for use in the treatment or prevention in a patient of a disease, disorder or symptom thereof delineated herein.

### Pharmaceutical Kits

The present invention also provides kits for use to treat epilepsy, Unverricht-Lundborg Disease (ULD) also known as progressive myoclonic epilepsy Type 1 (EPM1), tremor and symptomatic myoclonic epilepsies. These kits comprise (a) a pharmaceutical composition comprising a compound of Formula I or a salt thereof, wherein said pharmaceutical composition is in a container; and (b) instructions describing a method of using the pharmaceutical composition to treat epilepsy, Unverricht-Lundborg Disease (ULD) also known as progressive myoclonic epilepsy Type 1 (EPM1), tremor and symptomatic myoclonic epilepsies.

The container may be any vessel or other sealed or sealable apparatus that can hold said pharmaceutical composition. Examples include bottles, ampules, divided or multi-chambered holders bottles, wherein each division or chamber comprises a single dose of said composition, a divided foil packet wherein each division comprises a single dose of said composition, or a dispenser that dispenses single doses of said composition. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container employed can depend on the exact dosage form involved, for example a conventional cardboard box would not generally be used to hold a liquid suspension. It is feasible that more than one container can be used together in a single package to market a single dosage form. For example, tablets may be contained in a bottle, which is in turn contained within a box. In one embodiment, the container is a blister pack.

The kits of this invention may also comprise a device to administer or to measure out a unit dose of the pharmaceutical composition. Such device may include an inhaler if said composition is an inhalable composition; a syringe and needle if said composition is an injectable composition; a syringe, spoon, pump, or a vessel with or without volume markings if said composition is an oral liquid composition; or any other measuring or delivery device appropriate to the dosage formulation of the composition present in the kit.

In certain embodiments, the kits of this invention may comprise in a separate vessel of container a pharmaceutical composition comprising a second therapeutic agent, such as one of those listed above for use for co-administration with a compound of this invention.

### Example I

### Evaluation of Metabolic Stability

Microsomal Assay: Human liver microsomes (20 mg/mL) are obtained from Xenotech, LLC (Lenexa, Kans.). β-nicotinamide adenine dinucleotide phosphate, reduced form (NADPH), magnesium chloride (MgCl₂), and dimethyl sulfoxide (DMSO) are purchased from Sigma-Aldrich.

Determination of Metabolic Stability: 7.5 mM stock solutions of test compounds are prepared in DMSO. The 7.5 mM stock solutions are diluted to 12.5-50 μM in acetonitrile (ACN). The 20 mg/mL human liver microsomes are diluted to 0.625 mg/mL in 0.1 M potassium phosphate buffer, pH 7.4, containing 3 mM MgCl₂. The diluted microsomes are added to wells of a 96-well deep-well polypropylene plate in triplicate. A 10 μL aliquot of the 12.5-50 μM test compound is added to the microsomes and the mixture is pre-warmed for 10 minutes. Reactions are initiated by addition of pre-warmed NADPH solution. The final reaction volume is 0.5 mL and contains 0.5 mg/mL human liver microsomes, 0.25-1.0 μM test compound, and 2 mM NADPH in 0.1 M potassium phosphate buffer, pH 7.4, and 3 mM MgCl₂. The reaction mixtures are incubated at 37° C., and 50 μL aliquots are removed at 0, 5, 10, 20, and 30 minutes and added to shallow-well 96-well plates which contain 50 μL of ice-cold ACN with internal standard to stop the reactions. The plates are stored at 4° C. for 20 minutes after which 100 μL of water is added to the wells of the plate before centrifugation to pellet precipitated proteins. Supernatants are transferred to another 96-well plate and analyzed for amounts of parent remaining by LC-MS/MS using an Applied Bio-systems API 4000 mass spectrometer. The same procedure is followed for brivaracetam and the positive control, 7-ethoxycoumarin (1 μM). Testing is done in triplicate.

Data analysis: The in vitro t_{1/2}s for test compounds are calculated from the slopes of the linear regression of % parent remaining (ln) vs incubation time relationship.

in vitro t_{1/2}=0.693/k

k=−[slope of linear regression of % parent remaining (ln) vs incubation time]

Data analysis is performed using Microsoft Excel Software.

Without further description, it is believed that one of ordinary skill in the art can, using the preceding description and the illustrative examples, make and utilize the compounds of the present invention and practice the claimed methods. It should be understood that the foregoing discussion and examples merely present a detailed description of certain preferred embodiments. It will be apparent to those of ordinary skill in the art that various modifications and equivalents can be made without departing from the spirit and scope of the invention. All the patents, journal articles and other documents discussed or cited above are herein incorporated by reference.

We claim:

1. A compound of Formula I: 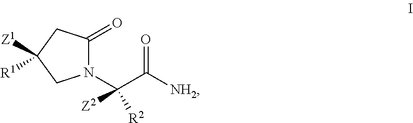
or a pharmaceutically acceptable salt thereof, wherein:
each Z is independently selected from hydrogen and deuterium;
R¹ is an n-propyl group having zero to seven deuterium atoms;
R² is an ethyl group having zero to five deuterium atoms; and
when each R has zero deuterium atoms, at least one Z is deuterium.

2. The compound of claim 1, wherein R¹ is selected from CD₃CH₂CH₂—, CD₃CD₂CH₂—, CD₃CH₂CD₂-, CH₃CH₂CD₂-, CH₃CD₂CD₂-, CD₃CD₂CD₂- or CH₃CH₂CH₂—.

3. The compound of claim 2, wherein R¹ is selected CD₃CD₂CD₂- or CD₃CD₂CH₂—.

4. The compound of any one of claims 1 to 3, wherein R² is selected from CH₃CH₂—, CD₃CH₂—, CH₃CD₂-, or CD₃CD₂-.

5. The compound of claim 4 wherein R² is selected from CH₃CH₂— or CD₃CD₂-.

6. The compound of claim 1 selected from any one of:

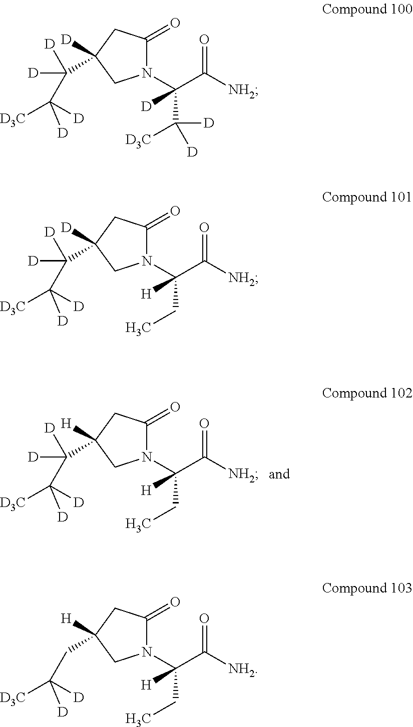

7. The compound of any one of claims 1 to 6, wherein any atom not designated as deuterium is present at its natural isotopic abundance.

8. A pyrogen-free pharmaceutically acceptable composition comprising a compound of claim 1 or a pharmaceutically acceptable salt thereof and a pharmaceutically acceptable carrier.

9. The composition of claim 8, further comprising a second therapeutic agent useful in treating a patient suffering from or susceptible to a disease or condition selected from epilepsy, epileptogenesis, seizure disorders, convulsions, bipolar disorders, mania, depression, anxiety, migraine, trigeminal and other neuralgia, chronic pain, neuropathic pain, cerebral ischemia, cardiac arrhythmia, myotonia, cocaine abuse, stroke, myoclonus, essential tremor and other movement disorders, neonatal cerebral hemorrhage, amyotrophic lateral sclerosis, spasticity, Parkinson's disease and other degenerative diseases, bronchial asthma, asthmatic status and allergic bronchitis, asthmatic syndrome, bronchial hyperreactivity, bronchospastic syndromes, allergic and vasomotor rhinitis, rhinoconjunctivitis, lower urinary tract dysfunction including urinary incontinence and overactive bladder, fecal incontinence, conditions of lower urinary tract dysfunction related to benign prostatic hyperplasia and fecal incontinence, and progressive myoclonic epilepsies (PME) including Unverricht-Lundborg disease, Lafora disease, myoclonic epilepsy with ragged red fibres, neuronal ceroid lipofuscinosis, sialidoses, and dentatorubral-pallidoluysian atrophy.

10. The composition of claim 9, wherein the second therapeutic agent is selected from carbamazepine, lamotrigine, levetiracetam, oxcarbazepine, phenyloin, topiramate, and valproate.

11. A composition for use in modulating the activity of synaptic vesicle protein 2A (SV2A) and/or inhibiting activity at neuronal voltage-dependent sodium channels in a cell of the central nervous system, the composition comprising a compound of claim 1 or a pharmaceutically acceptable salt thereof.

12. The composition of claim 8 for use in treating a disease or condition selected from epilepsy, epileptogenesis, seizure disorders, convulsions, bipolar disorders, mania, depression, anxiety, migraine, trigeminal and other neuralgia, chronic pain, neuropathic pain, cerebral ischemia, cardiac arrhythmia, myotonia, cocaine abuse, stroke, myoclonus, essential tremor and other movement disorders, neonatal cerebral hemorrhage, amyotrophic lateral sclerosis, spasticity, Parkinson's disease and other degenerative diseases, bronchial asthma, asthmatic status and allergic bronchitis, asthmatic syndrome, bronchial hyperreactivity, bronchospastic syndromes, allergic and vasomotor rhinitis, rhinoconjunctivitis, lower urinary tract dysfunction including urinary incontinence and overactive bladder, fecal incontinence, conditions of lower urinary tract dysfunction related to benign prostatic hyperplasia and fecal incontinence, and progressive myoclonic epilepsies (PME) including Unverricht-Lundborg disease, Lafora disease, myoclonic epilepsy with ragged red fibres, neuronal ceroid lipofuscinosis, sialidoses, and dentatorubral-pallidoluysian atrophy.

13. The composition of claim 12, wherein the disease or condition is selected from epilepsy, Unverricht-Lundborg Disease (ULD) also known as progressive myoclonic epilepsy Type 1 (EPM1), tremor and symptomatic myoclonic epilepsies.
